(19) United States
(12) Patent Application Publication
(10) Pub. No.: US 2010/0317919 A1
(43) Pub. Date: Dec. 16, 2010

(54) SPECTRAL ENDOSCOPE AND ITS WAVELENGTH CALIBRATION METHOD

(75) Inventors: Hideyuki Takaoka, Tokyo (JP); Shinya Matsumoto, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/446,304

(22) PCT Filed: Oct. 22, 2007
(86) PCT No.: PCT/JP2007/070536

§ 371 Date: Apr. 20, 2009

(30) Foreign Application Priority Data
Oct. 23, 2006 (JP) 2006-287438

(51) Int. Cl. A61B 1/04

(52) U.S. Cl. 600/104

(57) ABSTRACT

When using a spectral endoscope, the spectral characteristic can be precisely set to be suitable for use conditions. There is provided a spectral endoscope (1) comprising: a channel (3) arranged along a longitudinal direction in an insertion unit to be inserted into a body cavity; an image pickup section (8) which captures an image in a vicinity of the distal end of the insertion unit (2); a variable spectral section (7) capable of changing the wavelength of light to be incident into the image pickup section (8); a reference light member (10) which emits light of a known wavelength characteristic, or has a known absorption characteristic, and is to be introduced into a field-of-view range of the image pickup section (8) through the channel (3); a control unit (11) which controls the image pickup section (8) to capture an image of the reference light member (10) that has been introduced through the channel (3), while controlling the variable spectral section (7) to change the wavelength of light to be incident into the image pickup section (8); and a calibration unit (12) which calibrates the spectral characteristic of the variable spectral section (7) according to the image of the reference light member (10) that has been captured by the image pickup section (8).

### TECHNICAL FIELD

The present invention relates to a spectral endoscope and its wavelength calibration method.

### BACKGROUND ART

Conventionally, there is known a Fabry-Perot type variable spectral device which changes the face-to-face spacing between two planar optical substrates so as to change the wavelength of light to be transmitted therethrough (for example, refer to Patent Document 1).

This variable spectral device comprises reflection films and capacitance sensor electrodes on opposite surfaces of the respective optical substrates, so that the spacing dimension between the optical substrates is detected based on the capacitance value between the capacitance sensor electrodes, and the spacing between the optical substrates is changed by driving actuators to change the wavelength of light to be transmitted therethrough.

In addition, conventionally, there is also known a spectral endoscope comprising, in the distal end thereof, a built-in Fabry-Perot type variable spectral device that changes the face-to-face spacing between two planar optical substrates to change the wavelength of light to be transmitted therethrough (for example, refer to Patent Document 2).

According to this spectral endoscope, light in a specific wavelength band from the observation target can be selectively captured for imaging by transmitting light of a previously determined wavelength according to the face-to-face spacing between the optical substrates of the variable spectral device. Accordingly, imaging can be carried out while transmitting light of a desired wavelength by controlling the face-to-face spacing between the optical substrates.

Patent Document 1:

Japanese Unexamined Patent Application, Publication No. 2002-277758

Patent Document 2:

Japanese Unexamined Patent Application, Publication No. 2006-25802

### DISCLOSURE OF INVENTION

A spectral endoscope is inserted and disposed in a body cavity of the patient for use in diagnosis and treatment, and thus has to be used in a different environment from environments where it was manufactured or stored. Inside of the body cavity of the patient is highly humid with a temperature of about 36° C., differing from the ambient humidity and temperature outside the body of the patient. Generally, the insertion unit of an endoscope has a watertight structure; however, when inserted into a body cavity of the patient, the temperature and the humidity in a space between the optical substrates of the variable spectral device built in the spectral endoscope, are slightly changed. This shows that the refractive index and the permittivity of the air between the optical substrates of the variable spectral device fluctuate, and thereby the transmission wavelength also fluctuates. Accordingly, even if precise calibration has been done outside the body, the spectral characteristic is changed in the body cavity. Because of this, inconveniently, imaging of light in a desired wavelength band can not be performed.

The present invention takes the above situation into consideration with an object of providing a spectral endoscope and its wavelength calibration method by which the spectral characteristic can be precisely set to be suitable for use conditions of the spectral endoscope.

In order to achieve the above object, the present invention provides the following solutions.

A first aspect of the present invention is a spectral endoscope comprising: a channel arranged along a longitudinal direction in an insertion unit to be inserted into a body cavity; an image pickup section which captures an image in a vicinity of the distal end of the insertion unit; a variable spectral section capable of changing the wavelength of light to be incident into the image pickup section; a reference light member which emits light of a known wavelength characteristic, or has a known absorption characteristic, and is to be introduced into a field-of-view range of the image pickup section through the channel; a control unit which controls the image pickup section to capture an image of the reference light member that has been introduced through the channel, while controlling the variable spectral section to change the wavelength of light to be incident into the image pickup section; and a calibration unit which calibrates the spectral characteristic of the variable spectral section according to the image of the reference light member that has been captured by the image pickup section.

In the first aspect, said image pickup section and said variable spectral section may be arranged in the distal end of said insertion unit.

In addition, in the first aspect, said reference light member may comprise a fluorescent substance which generates fluorescence of a known wavelength characteristic by excitation light emitted from the distal end of said insertion unit.

Moreover, in the first aspect, said reference light member may comprise a reflection member having a known reflection spectrum with respect to illumination light emitted from the distal end of said insertion unit.

Furthermore, in the first aspect, said reference light member may comprise a light source which generates light of a known wavelength characteristic.

In addition, in the first aspect, the wavelength of light to be incident into said image pickup section may be continuously scanned by said variable spectral section so that said image pickup section can continuously capture the image of said reference light member.

Moreover, in the first aspect, the wavelength characteristic of said reference light member may have a narrow-band peak at a specific wavelength. In this case, the wavelength characteristic of said reference light member may have a plurality of narrow-band peaks.

Furthermore, in the first aspect, the wavelength band of light emitted from or absorbed into said reference light member may be approximately the same as a wavelength band of fluorescence generated from a fluorescent agent to be administered at the time of observation.

In addition, a second aspect of the present invention is a spectral endoscope comprising: a channel arranged along a longitudinal direction in an insertion unit to be inserted into a body cavity; an image pickup section which captures an image in a vicinity of the distal end of the insertion unit; a variable spectral section capable of changing the wavelength of light to be incident into the image pickup section; a control unit which controls the image pickup section to capture an image of a reference light member which emits light of a known wavelength characteristic, or has a known absorption characteristic, and is to be introduced into a field-of-view range of the image pickup section through the channel, while controlling the variable spectral section to change the wavelength of light to be incident into the image pickup section; and a calibration unit which calibrates the spectral characteristic of the variable spectral section according to the image of the reference light member that has been captured by the image pickup section.

Moreover, a third aspect of the present invention is a reference light member which emits light of a known wavelength characteristic, or has a known absorption characteristic, and is to be introduced into a field-of-view range of an image pickup section which captures an image in a vicinity of the distal end of an insertion unit of an spectral endoscope through a channel arranged along a longitudinal direction in the insertion unit, for use in calibration of a spectral characteristic of a variable spectral section capable of changing the wavelength of light to be incident into the image pickup section.

Furthermore, a fourth aspect of the present invention is a wavelength calibration method for a spectral endoscope comprising: a channel arranged along a longitudinal direction in an insertion unit to be inserted into a body cavity; an image pickup section which captures an image in a vicinity of the distal end of the insertion unit; and a variable spectral section capable of changing the wavelength of light to be incident into the image pickup section, wherein the method comprises: a step of introducing a reference light member which emits light of a known wavelength characteristic, or has a known absorption characteristic, into a field-of-view range of the image pickup section through the channel; a step of controlling the image pickup section to capture an image of the reference light member, while controlling the variable spectral section to change the wavelength of light to be incident into the image pickup section; and a step of calibrating the spectral characteristic of the variable spectral section according to the image of the reference light member that has been captured by the image pickup section.

The present invention demonstrates an effect in which the spectral characteristic can be precisely set to be suitable for use conditions of the spectral endoscope.

### BRIEF DESCRIPTION OF DRAWINGS

FIG. 1 is a schematic diagram showing the distal end of a spectral endoscope according to one embodiment of the present invention.

FIG. 2 shows an example of an image including a reference fluorescence member captured by the spectral endoscope of FIG. 1.

FIG. 3 is a flowchart showing a wavelength calibration method according to one embodiment of the present invention in which the spectral endoscope of FIG. 1 is used.

FIG. 4 shows an example of the wavelength characteristic of the reference fluorescence member for use in the wavelength calibration method of FIG. 3.

FIG. 5 shows the relationship between the detected signal Vs of the capacitance sensor and the fluorescence intensity of the reference fluorescence member that have been measured by using the reference fluorescence member of FIG. 4.

### EXPLANATION OF REFERENCE SIGNS

1: Spectral endoscope
2: Insertion unit
3: Forceps channel (Channel)
7: Variable spectral device (Variable spectral section)
8: CCD (Image pickup section)
10: Reference fluorescence member (Reference light member)
11: Control unit
12: Calibration unit

### BEST MODE FOR CARRYING OUT THE INVENTION

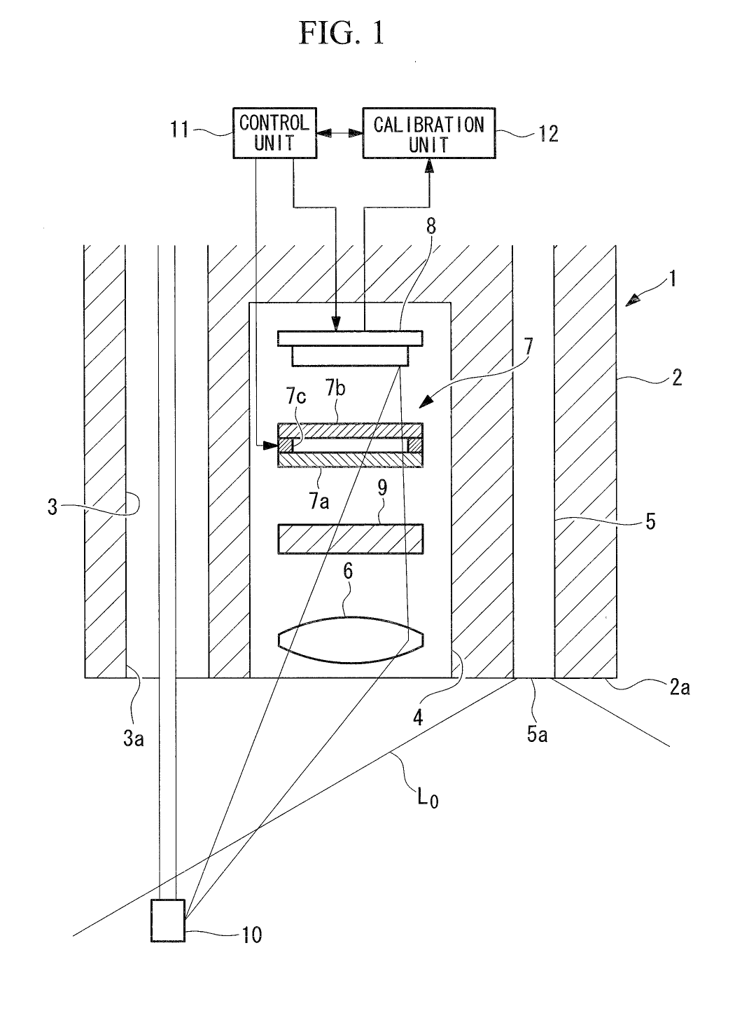

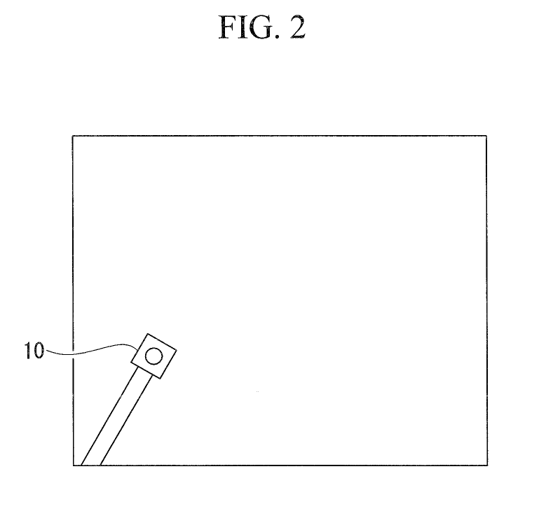

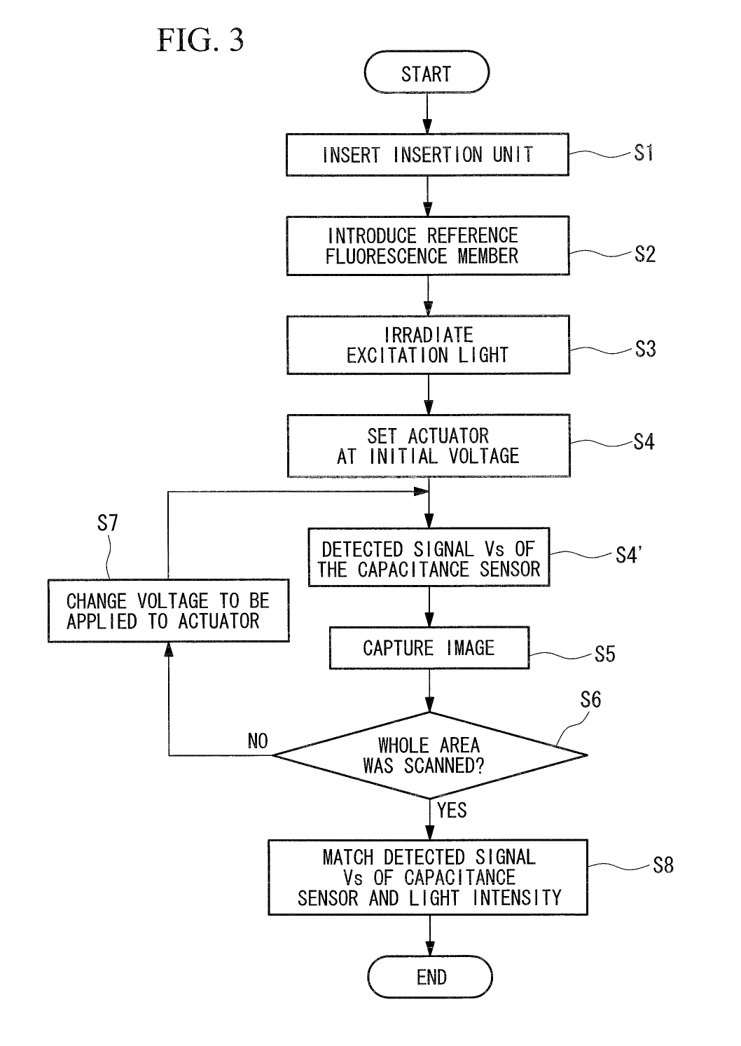

Hereunder is a description of a spectral endoscope 1 and its wavelength calibration method according to one embodiment of the present invention, with reference to FIG. 1 to FIG. 5.

As shown in FIG. 1, the spectral endoscope 1 to which the wavelength calibration method according to this embodiment is applied, comprises a long and slender insertion unit 2 to be inserted into a body cavity of the patient. In the insertion unit 2 is arranged a forceps channel (channel) 3 for insertion of treatment tools such as forceps, along an approximately all over the longitudinal direction of the insertion unit 2.

In addition, on the distal end of the insertion unit 2 are arranged one end faces of an imaging unit 4 and a light guide (excitation light emission unit) 5 which emits excitation light L₀.

The imaging unit 4 comprises an object lens 6 which converges light incident from the forward area beyond the distal end face 2a of the insertion unit 2, a variable spectral device (variable spectral section) 7 which spectrally disperses the light converged by the object lens 6, and a CCD (image pickup section) 8 which captures the image of light passing through the variable spectral device 7. In the drawing, the reference sign 9 denotes an excitation light cut filter which cuts off excitation light L₀ of a predetermined wavelength that has been converged by the object lens 6, the reference sign 10 denotes a control unit which controls the variable spectral device 7 and the CCD 8, and the reference sign 11 denotes a calibration unit.

As shown in FIG. 1, the variable spectral device 7 comprises: two optical substrates 7a and 7b arranged with a parallel spacing; actuators 7c such as a piezoelectric element which is arranged between these optical substrates 7a and 7b, and is driven to adjust the spacing dimension between these two optical substrates 7a and 7b; and a capacitance sensor (not shown) which comprises electrodes made of metal films that are arranged in respectively opposed positions on opposite surfaces of these two optical substrates 7a and 7b.

The control unit 11 controls the actuators 7c and the CCD 8 on the basis of a signal from the capacitance sensor. By changing the voltage to be applied to the actuators 7c, the actuators 7c is extended or contracted to change the spacing dimension between the optical substrates 7a and 7b. In addition, at this time, on the basis of the detected signal of the capacitance sensor, the spacing dimension between the optical substrates 7a and 7b is detected, so that the relational equation (1) between this spacing dimension and the transmission wavelength characteristic can be used to achieve the feedback control of the voltage to be applied to the actuators 7c.

As shown in equation (1), Fabry-Perot type variable spectral devices are capable of selective acquisition of periodic transmission spectral peaks on the basis of wavelength λ which resonates with the face-to-face spacing d between a pair of reflection films, because of the light interference effect.

2nd cos θ=mλ (1), wherein

n: refractive index of a medium filling the face-to-face spacing d between a pair of reflection films (n=1 when the medium is air)

d: face-to-face spacing between a pair of reflection films

λ: wavelength

θ: incidence angle into reflection films

m: order (integral number)

According to this spectral endoscope 1, by emitting excitation light L₀ from the distal end face 5a of the light guide 5, a fluorescent substance within a living body (not shown) serving as the observation target is excited to generate fluorescence. The fluorescence is converged by the object lens 6, and spectrally dispersed by the variable spectral device 7, and its image is captured by the CCD 8. The excitation light L₀ that has been reflected and is returning within the living body is cut off by the excitation light cut filter 9 and thus is not incident into the CCD 8.

In addition, through the variable spectral device 7, among the fluorescence incident into the imaging unit 4, fluorescence in a predetermined wavelength band is exclusively allowed to enter the CCD 8. That is to say, the operation of the variable spectral device 7 enables selection in accordance with the purpose, of fluorescence generated by excitation with the excitation light L₀, from either a fluorescent agent that has been introduced into the living body, or an autofluorescent substance that has been originally present within the living body, and enables capture of the image of the thus selected fluorescence.

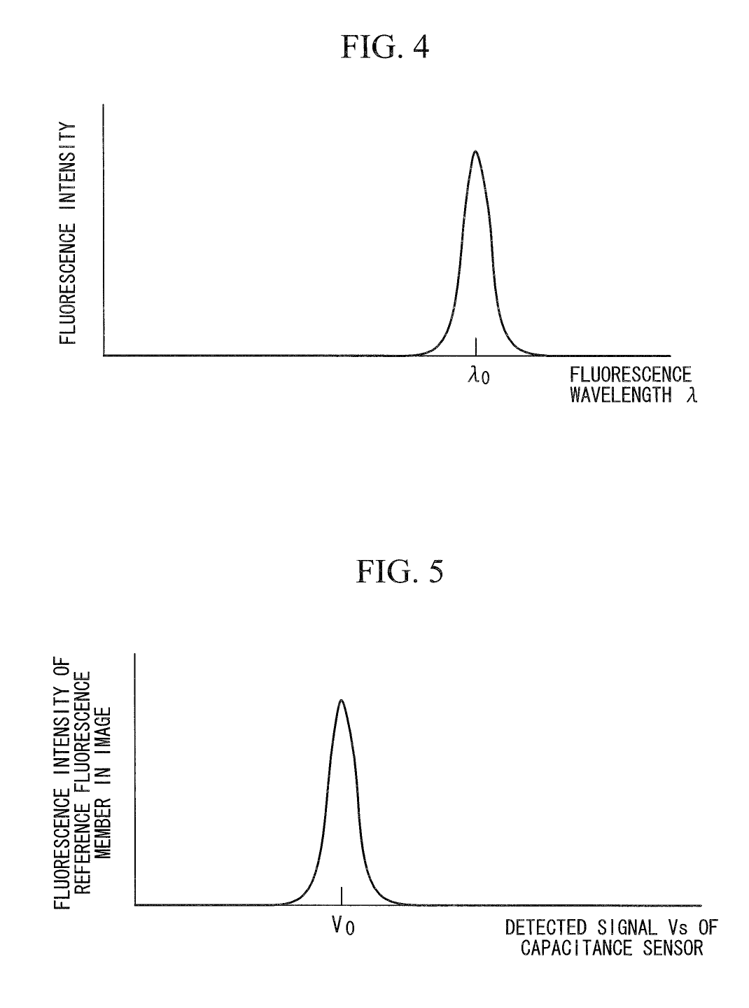

In the wavelength calibration method for the spectral endoscope 1 according to this embodiment, first, the insertion unit 2 of the spectral endoscope 1 is inserted into the body cavity, and the distal end thereof is disposed in a desired location (Step S1). In this state, as shown in FIG. 1, the reference fluorescence member (reference light member) 10 is introduced into the body cavity through the forceps channel 3 of the insertion unit 2 (Step S2). The distal end thereof is arranged in a field-of-view range of the imaging unit 4. The reference fluorescence member 10 has a distal end to be projected from the distal opening 3a of the forceps channel 3, being coated with a fluorescent substance which generates fluorescence L₁ of a known wavelength characteristic by excitation with excitation light L₀ emitted from the distal end face 5a of the light guide 5. For example, as shown in FIG. 4, such a fluorescent substance has a wavelength characteristic having a single peak at a certain wavelength λ₀.

Next, excitation light L₀ is emitted from the distal end face 5a of the light guide 5 (Step S3). By so doing, the emitted excitation light L₀ is irradiated onto the reference fluorescence member 10 being projected forward from the insertion unit, and the fluorescent substance on the reference fluorescence member 10 is excited to generate fluorescence L₁. Since the thus generated fluorescence L₁ has a known wavelength characteristic, this wavelength characteristic is measured. Through this measurement, the detected signal Vs of the capacitance sensor and the wavelength λ of the fluorescence L₁ transmitting through the variable spectral device 7 can be accurately matched (calibrated). By so doing, the control unit can be calibrated so as to realize the accurate feedback control of the voltage to be applied to the actuator 7c.

Specifically, the control unit 11 sets the initial voltage V to be applied to the actuator 7 (Step S4). Next, while changing the voltage V to be applied to the actuator 7c so that, for example, the wavelength of light to be transmitted can be changed continuously from the short wavelength side to the long wavelength side, the detected signal Vs of the capacitance sensor and the light intensity of the image of the reference fluorescence member 10 captured by the CCD 8 are detected (Step S4′ to Step S7). By so doing, as shown in FIG. 5, the relationship between the detected signal Vs of the capacitance sensor and the light intensity of the image of the reference fluorescence member 10 captured by the CCD 8 can be obtained (Step S8). According to this relationship graph, when the detected signal Vs of the capacitance sensor is V₀, the light intensity of the image of the reference fluorescence member 10 reaches its peak. Accordingly, it can be understood that the variable spectral device 7 at this time is in the state for transmitting fluorescence L₁ of a wavelength λ₀, by which the spacing dimension between the optical substrates 7a and 7b, calculated from the detected value of the capacitance sensor and the transmission wavelength characteristic, can be accurately matched. By so doing, therefore, the control unit can be calibrated, as a result of which the spectral characteristic of the variable spectral device 7 can be calibrated.

In this manner, according to the wavelength calibration method for the spectral endoscope 1 of this embodiment, the variable spectral device 7 can be calibrated in a state where the insertion unit 2 of the spectral endoscope 1 is being inserted in the body cavity and the distal end thereof is being disposed in the vicinity of the observation target. As a result, an advantage will be given in which the spectral characteristic of the variable spectral device 7, even if changed due to the variation of the ambient humidity and temperature surrounding the insertion unit 2, can be precisely calibrated, so that a sharp fluorescence image can be obtained through highly precise spectral dispersion for desired fluorescence L₁.

As for the reference fluorescence member 10 in this embodiment, it is preferable to employ a member which generates fluorescence L₁ in an approximately same wavelength band as the wavelength band of fluorescence generated by the fluorescent agent to be used for the observation of the observation target. By so doing, the spectral characteristic of the variable spectral device 7 can be calibrated within the wavelength band to be used for the actual observation, and a fluorescence image can be obtained through more precise spectral dispersion.

In addition, as for the reference fluorescence member 10, there may also be employed a treatment tool (not shown) to be inserted through the forceps channel 3, the distal end of which is coated with a fluorescent substance.

In the wavelength calibration method for the spectral endoscope 1 according to this embodiment, such a case has been exemplified in which the reference fluorescence member 10 is coated with a fluorescent substance of a wavelength characteristic having a single narrow-band peak; however, instead of this, there may also be employed a member coated with a fluorescent substance of a wavelength characteristic having a plurality of narrow-band peaks, or a plurality of fluorescent substances of wavelength characteristics having different single narrow-band peaks. By so doing, the precision of calibration regarding the spectral characteristic of the variable spectral device 7 can be improved.

In addition, as for reference light member, the reference fluorescence member 10 coated with a fluorescent substance which generates fluorescence L₁ of a known wavelength characteristic by excitation with excitation light L₀, has been exemplified; however, instead of this, it is also possible to employ a reference reflection member (not shown) which has a known wavelength characteristic and reflects light in a predetermined wavelength band, and to emit illumination light from the light guide. Also by so doing, similarly to the above embodiment, while changing the voltage V to be applied to the actuator 7c of the variable spectral device 7, light that has been reflected on the reference reflection member and is returning, can be detected; by which, the relationship between the detected signal Vs of the capacitance sensor and the transmission wavelength characteristic can be accurately calibrated, and thus the spectral characteristic of the variable spectral device 7 can be precisely calibrated.

Furthermore, instead of such a reference reflection member, there may also be employed a reference absorption member which absorbs light in a predetermined wavelength band.

Moreover, as for the reference light member, it is also possible to employ a light source which generates light of a predetermined wavelength by itself, or either an optical fiber or a light guide which transmits light generated from such a light source and emits it from the distal end thereof, so as to perform calibration without emitting light from the light guide 5a provided in the insertion unit 2 of the spectral endoscope 1.

In addition, as for the reference light member, it is also possible to employ a fluorescent agent having a known wavelength characteristic and to spray this fluorescent agent over areas except for the site of the observation target within the body cavity; so that, while irradiating the excitation light L₀ thereon and changing the voltage V to be applied to the actuator 7c of the variable spectral device 7, the detected signal Vs of the capacitance sensor and thus generated fluorescence L₁ can be detected so as to thereby, similarly to the above embodiment, calibrate the spectral characteristic of the variable spectral device 7. Furthermore, similar calibration can be performed by spraying a fluorescent agent of a wavelength characteristic having a narrow-band peak in a wavelength band that is sufficiently apart from the wavelength band to be used for the observation, over the vicinity of the site of the observation target.

1. A spectral endoscope comprising: a channel arranged along a longitudinal direction in an insertion unit to be inserted into a body cavity;
an image pickup section which captures an image in a vicinity of the distal end of the insertion unit;
a variable spectral section capable of changing a wavelength of light to be incident into the image pickup section;
a reference light member which emits light of a known wavelength characteristic, or has a known absorption characteristic, and is to be introduced into a field-of-view range of the image pickup section through the channel;
a control unit which controls the image pickup section to capture an image of the reference light member that has been introduced through the channel, while controlling the variable spectral section to change the wavelength of light to be incident into the image pickup section; and
a calibration unit which calibrates a spectral characteristic of the variable spectral section according to the image of the reference light member that has been captured by the image pickup section.

2. A spectral endoscope according to claim 1, wherein said image pickup section and said variable spectral section are arranged in the distal end of said insertion unit.

3. A spectral endoscope according to claim 1, wherein said reference light member comprises a fluorescent substance which generates fluorescence of a known wavelength characteristic by excitation light emitted from the distal end of said insertion unit.

4. A spectral endoscope according to claim 1, wherein said reference light member comprises a reflection member having a known reflection spectrum with respect to illumination light emitted from the distal end of said insertion unit.

5. A spectral endoscope according to claim 1, wherein said reference light member comprises a light source which generates light of a known wavelength characteristic.

6. A spectral endoscope according to claim 1, wherein the wavelength of light to be incident into said image pickup section is continuously scanned by said variable spectral section so that said image pickup section can continuously capture the image of said reference light member.

7. A spectral endoscope according to claim 1, wherein the wavelength characteristic of said reference light member has a narrow-band peak at a specific wavelength.

8. A spectral endoscope according to claim 7, wherein the wavelength characteristic of said reference light member has a plurality of narrow-band peaks.

9. A spectral endoscope according to claim 1, wherein the wavelength band of light emitted from or absorbed into said reference light member is approximately the same as a wavelength band of fluorescence generated from a fluorescent agent to be administered at the time of observation.

10. A spectral endoscope comprising: a channel arranged along a longitudinal direction in an insertion unit to be inserted into a body cavity;
an image pickup section which captures an image in a vicinity of the distal end of the insertion unit;
a variable spectral section capable of changing a wavelength of light to be incident into the image pickup section;
a control unit which controls the image pickup section to capture an image of a reference light member which emits light of a known wavelength characteristic, or has a known absorption characteristic, and is to be introduced into a field-of-view range of the image pickup section through the channel, while controlling the variable spectral section to change the wavelength of light to be incident into the image pickup section; and
a calibration unit which calibrates a spectral characteristic of the variable spectral section according to the image of the reference light member that has been captured by the image pickup section.

11. A reference light member which emits light of a known wavelength characteristic, or has a known absorption characteristic, and is to be introduced into a field-of-view range of an image pickup section which captures an image in a vicinity of the distal end of an insertion unit of an spectral endoscope through a channel arranged along a longitudinal direction in the insertion unit, for use in calibration of a spectral characteristic of a variable spectral section capable of changing a wavelength of light to be incident into the image pickup section.

12. A wavelength calibration method for a spectral endoscope comprising: a channel arranged along a longitudinal direction in an insertion unit to be inserted into a body cavity; an image pickup section which captures an image in a vicinity of the distal end of the insertion unit; and a variable spectral section capable of changing a wavelength of light to be incident into the image pickup section, wherein
the method comprises: a step of introducing a reference light member which emits light of a known wavelength characteristic, or has a known absorption characteristic, into a field-of-view range of the image pickup section through the channel;
a step of controlling the image pickup section to capture an image of the reference light member, while controlling the variable spectral section to change the wavelength of light to be incident into the image pickup section; and
a step of calibrating a spectral characteristic of the variable spectral section according to the image of the reference light member that has been captured by the image pickup section.
